(19) United States
(12) Patent Application Publication
(10) Pub. No.: US 2022/0001084 A1
(43) Pub. Date: Jan. 6, 2022

(54) COMPOSITE STINT APPARATUS AND FABRICATION METHOD

(71) Applicant: TKO Discovery, Inc., Knoxville, TN (US)

(72) Inventors: Mira Baraket, Knoxville, TN (US); William A. Malkes, Knoxville, TN (US); Shannon Eaker, Knoxville, TN (US); James Vig Sherrill, Knoxville, TN (US)

(21) Appl. No.: 17/367,070

(22) Filed: Jul. 2, 2021

Related U.S. Application Data
(60) Provisional application No. 63/047,345, filed on Jul. 2, 2020.

(51) Int. Cl. A61L 31/02; A61L 31/08; C08L 75/04; C08L 27/18; A61L 31/06; A61L 31/04; A61L 31/16

(52) U.S. Cl. CPC A61L 31/024; A61L 31/022; A61L 31/084; C08L 75/04; A61L 2400/18; A61L 31/06; A61L 31/048; A61L 31/16; A61L 2420/08; C08L 27/18

(57) ABSTRACT

The present disclosure is generally related to a stent with one more layer of graphene added to a stint that will be inserted into the body of a patient. Such a stint may be inserted into a vein or artery of a patient in order to increase blood flow, to maintain blood flow, or to prevent a vein or artery from collapsing. The graphene added to the stint provides improved biocompatibility of the stint and reduces risks associated with conventional stints. Stints consistent with the present disclosure may also include growth factors that help bind the stint to specific receptors or target cells. Stints of the present disclosure may include a monolayer, a bilayer, or multi-layers of graphene adhered to the surface of the stent. The stint may then coated with a growth factor.

### CROSS-REFERENCE TO RELATED APPLICATIONS

The present disclosure claims priority benefit of U.S. provisional patent application 63/047,345 filed Jul. 2, 2020, the disclosure of which is incorporated by reference herein.

### BACKGROUND

### Field of the Disclosure

The present disclosure is generally directed to improved stints. More specifically, the present disclosure is directed to stints that made of or that include material improvements.

### Description of the Related Art

A medical stent is generally defined as a tubular structure and support that is placed or inserted inside a blood vessel, canal, or duct to keep the passageway open usually to relieve blockages or obstructions or aid in healing. There are a wide variety of stents that are used for different applications or purposes, such as, expandable coronary, vascular, biliary stents, and plastics stents for urinary, kidney, and bladder applications. Stents can typically be made up of a metal or polymer (i.e. plastic)

Many types of stents are well known in the art, such as, coronary stents that are placed during coronary angioplasty, vascular stents that are commonly placed during peripheral artery angioplasty, a stent graft or covered stent is vascular stent with fabric coating that creates an expandable tube, ureteral stents, prostatic stents, esophageal stents, biliary stents, glaucoma drainage stents, duodenal stents, colonic stents, and pancreatic stents. “Stent” or “Stenting” is also used to describe the placement of such a device.

Drug-eluting stents are also well known in the art. As many stents are made of materials that don't absorb other materials easily the stent then does not care drugs coated on them very well or for any period of time. Some stents require “sleeves” often made of polymeric materials that can control the release of a therapeutic drug. Often times these drugs are required to prevent side effects caused by the metallic or polymeric construction of the stent, such as rejection or allergic reaction to the materials.

Currently, stents are constructed of either a metal alloy or polymer which can cause the body to have an allergic reaction and reject the stent. The reaction and rejection can cause significant side effects such as Kounis syndrome and further require additional medication to prevent the rejection or further surgery to replace the stent. Another concern with stent and stent placement is getting the right cells to the location or spot to create the endothelial layer instead of getting other cells or muscles cells. Clot formation or clotting is another risk or concern associated with stents and stent placement and there is a big need to reduce the risk. Furthermore, sites were stints are place commonly suffer from a condition referred to as narrowing. This narrowing can limit the utility of stints. There is a need to develop new stints that mitigate or eliminate the possibility of inducing an allergic reaction in patients. There is a need to provide stints that help create an endothelial layer and that may help reduce the likelihood of clot creation. There is also a need to improve stints by making them smaller, stronger, or both smaller and stronger.

### SUMMARY OF THE PRESENTLY CLAIMED INVENTION

The presently claimed invention is directed to an improved stint and to methods for making improved stints. In one embodiment a stint that may be inserted into the body of a person includes a base material that may be included in the structure of the stint. This stint may include a biocompatible graphene that is deposited on to a surface of the structure according to a rule. The structure of the stint may be capable of expanding to a larger size from a compressed size after being inserted into a body of a patient and the stint may be received by the body of the patient based on the biocompatible graphene being deposited according to the rule.

Another embodiment of the present disclosure is directed to a method for making a stint. Here the method may include preparing a base material to deposit graphene onto, identifying a method for depositing the graphene onto a portion of the base material via the identified method according to the rule and depositing the graphene onto the graphene onto the portion of the base material via the identified method according to the rule.

### BRIEF DESCRIPTIONS OF THE DRAWINGS

FIG. 1 is an apparatus for a stent with layer or layers of graphene and growth factors.

FIG. 2 illustrates structures of scaffold structures that may be used in different stints.

FIG. 3 illustrates a structure of a graphene layer that may be deposited on surfaces of a stint.

FIG. 4 illustrates a second cross-sectional or end view of a stint that is similar to the cross-sectional or end view 140 of stint 100 of FIG. 1

FIG. 5 illustrates a series of steps that may be performed when a stint consistent with the present disclosure is manufactured.

FIG. 6 illustrates several different measurements made by Raman spectroscopy.

### DETAILED DESCRIPTION

The present disclosure is generally related to a stent with one more layer of graphene added to a stint that will be inserted into the body of a patient. Such a stint may be inserted into a vein or artery of a patient in order to increase blood flow, to maintain blood flow, or to prevent a vein or artery from collapsing. The graphene added to the stint provides improved biocompatibility of the stint and reduces risks associated with conventional stints. Stints consistent with the present disclosure may also include growth factors that help bind the stint to specific receptors or target cells. Stints of the present disclosure may include a monolayer, a bilayer, or multi-layers of graphene adhered to the surface of the stent. The stint may then coated with a growth factor.

A stent for implantation into a body vessel in which a cylindrical stent body coiled from a generally continuous wire with a deformable zig-zag structure, where the sent is covered with a one or more layers of graphene and then the stint may be coated with a growth factor such as the NELL1 protein or Vascular endothelial growth factor (VEGF). A graphene coating can help reduce an allergic reaction or the rejection of the stent due to the metal alloy or polymer materials. Furthermore, the functionalization of a growth factor such as NELL1 or VEGF protein on graphene is much more direct than putting it on various metal alloys or polymers which are incompatible with a coating. Additionally, the graphene coating may coat the entire stent or portion of the stent depending on the application of the stent or a location where the stent in inserted.

Graphene materials of the present disclosure may be a one-atom-thick sheet of carbon atoms arranged in a honeycomb-like pattern. Graphene is considered to be the world's thinnest, strongest and most conductive material—to both electricity and heat. All this property are exciting researchers and businesses around the world—as graphene has the potential the revolutionize entire industries—in the fields of electricity, conductivity, energy generation, batteries, sensors and more. Graphene has a lot of other promising applications: anti-corrosion coatings and paints, efficient and precise sensors, faster and efficient electronics, flexible displays, efficient solar panels, faster DNA sequencing, drug delivery, and more. Graphene is such a great and basic building block that it seems that any industry can benefit from this new material. Time will tell where graphene will indeed make an impact—or whether other new materials will be more suitable.

Graphene is considered as having a good biocompatibility and biodegradability, these factors make it an attractive material for medical research in fields like medical tools and drug delivery carriers. However, a recurring issue is the difficulty in modifying many individual functional molecules onto a graphene nano-sheet at the same time for biomedical applications.

Various different methods may be used to manufacture and depositing graphene, for example when making a stent. One example including the chemical vapor deposition (CVD) process. CVD is a vacuum deposition method used to produce high quality, high-performance, solid materials. The process is often used in the semiconductor industry to produce thin films. In typical CVD, the wafer (substrate) is exposed to one or more volatile precursors, which react and/or decompose on the substrate surface to produce the desired deposit. Frequently, volatile by-products are also produced, which are removed by gas flow through the reaction chamber. Microfabrication processes widely use CVD to deposit materials in various forms, including monocrystalline, polycrystalline, amorphous, and epitaxial. These materials include silicon (dioxide, carbide, nitride, oxynitride), carbon (fiber, nanofibers, nanotubes, diamond, and graphene), fluorocarbons, filaments, tungsten, titanium nitride, and various high-k dielectrics. CVD.

Other methods of manufacturing for example may include, exfoliation produced graphene with the lowest number of defects and highest electron mobility. Epitaxy refers to the deposition of a crystalline overlayer on a crystalline substrate, where there is registry between the two. In some cases, epitaxial graphene layers are coupled to surfaces weakly enough to retain the two-dimensional electronic band structure of isolated graphene. Epitaxial graphene films can be grown on various crystalline surfaces. The atomic lattice of the substrate facilitate in orientationally registering the carbon atoms of the graphene layer. The chemical interaction of the graphene with the substrate can vary from weak to strong. Graphene can be created by cutting open carbon nanotubes. In one such method multi-walled carbon nanotubes are cut open in solution by action of potassium permanganate and sulfuric acid. In another method, graphene nanoribbons were produced by plasma etching of nanotubes partly embedded in a polymer film. In applications where the thickness and packing density of graphene layer needs to carefully controlled, the Langmuir-Blodgett method has been used. In addition to directly forming a layer of graphene, another approach that has been widely studied is forming a graphene oxide layer which can then be reduced further into graphene.

Another method includes a highly exothermic reaction combusts magnesium in an oxidation-reduction reaction with carbon dioxide, producing a variety of carbon nanoparticles including graphene and fullerenes. The carbon dioxide reactant may be either solid (dry-ice) or gaseous. The products of this reaction are carbon and magnesium oxide. carbon nanotube-reinforced graphene was made via spin coating and annealing functionalized carbon nanotubes. The resulting material was stronger, flexible, and more conductive than conventional graphene.

Supersonic acceleration of droplets through a Laval nozzle was used to deposit small droplets of reduced graphene-oxide in suspension on a substrate is another method. The droplets disperse evenly, evaporate rapidly, and display reduced flake aggregations. Producing graphene via intercalation splits graphite into single-layer graphene by inserting guest molecules/ions between the graphite layers. Another method includes a laser-based single-step, scalable approach to graphene production. The technique produces and patterns porous three-dimensional graphene film networks from commercial polymer films. The system used a CO2 infrared laser. The sp3-carbon atoms were photothermally converted to sp2-carbon atoms by pulsed laser irradiation.

A process of making a composite material that includes graphene and one or more polymers may include multiple transfer steps. Here, the graphene may be produced on a metallic substrate (e.g. copper) using chemical vapor deposition (CVD), and then be transferred to a polymer surface using various dry or wet transfer methods, including oxidative decoupling, electrochemical delamination and/or etching transfer methods. In the case of oxidative decoupling method, the graphene on metal surface (i.e. copper) may be exposed to oxidizing agents (water and/or other solvent) at temperatures ranging from 20 degrees Celsius (C) to 100 C, which permits the oxidation of copper at the Cu/graphene interface, the resulting graphene/copper oxide/graphene may be in a decoupled state that permits a graphene delamination a by hot lamination or hot press. This delamination of the surface coated with graphene that may be made easier when the graphene is in a decoupled state (e.g. decoupled graphene). The present disclosure is not limited to the process of depositing graphene onto a substrate that may be metallic as described above, yet is exemplary of processes that could be used to prepare graphene that may be in a decoupled form onto a substrate during a portion of a manufacturing process.

Once the decoupled graphene and metal, like the copper mentioned above is prepared, other steps may be performed to transfer the graphene onto a surface of a polymer. This process may also include the deposition of multiple layers of graphene onto a metallic substrate. The process of combining the decoupled graphene with the polymer may include making of a polymer substrate or layer onto which the decoupled graphene is transferred onto or into.

Exemplary polymers that may be used include, yet are not limited to polyurethane (PU), layers of PU and polytetrafluoroethylene (PTFE) or Polyvinylidene fluoride (PVDF), and layers of PU and expanded polytetrafluoroethylene (ePTFE). A process of creating a polymer surface or substrate may include depositing, chemical vapor deposition, or melt casting, or by solution-based processing: spin coating, film or solution casting, or spraying a thin layer of PU onto a previously manufactured or purchased sheet of ePTFE. This deposition or spraying process may include the use of a solvent that acts as a mechanism to transport PU in a solution after which the solvent may be evaporated. The present disclosure is not limited to the above-mentioned polymers and can be extended to others.

Once a polymer sheet and a metallic surface/substrate coated with decoupled graphene are prepared, a surface of the polymer and the side of the metallic substrate upon which the decoupled graphene may be pressed together. This may include applying heat that may help facilitate transfer of the graphene onto the polymer. Heat may be applied by various means, for example by a heated roller, a heated environment, a hot press, or combination thereof. Pressures in the range of 1 to 10,000 PSI may be used during this lamination/hot press process and this process may include temperatures in the range of 20 to 250 degrees C., for example. A low pressure range may be used for lamination onto a PU layer, while a high pressure range may be used for hot pressing onto PVDF, PTFE or ePTFE.

In the case of dry transfer method using the oxidative decoupling method, and After the graphene has been impressed onto or into the polymer, the metallic (e.g. copper) substrate may be removed by simply lifting the substrate away from the composite material of polymer and graphene. The process of removing the metallic substrate may be performed at or near the hot press temperature of may be performed at yet another temperature that may be colder or hotter than the hot press temperature. Here a change in temperature may help sheer the graphene away from the metallic substrate.

After the polymer and graphene composite material is made into a sheet, that sheet may be cut into pieces that are used to manufacture a stent. Such a stent may be made by knitting one or more strips of the composite material into an expandable tubular shape. Stents may be made from a spiral, a helical spiral, may be woven or knitted, or may be made as a series of sequential rings. The composite material may be cut using any method common in the art, such as laser cutting, for example.

A growth factor is a naturally occurring substance capable of stimulating cellular growth, proliferation, healing, and cellular differentiation. Usually, it is a protein or a steroid hormone. Growth factors are important for regulating a variety of cellular processes. Growth factors typically act as signaling molecules between cells. Examples are cytokines and hormones that bind to specific receptors on the surface of their target cells. They often promote cell differentiation and maturation, which varies between growth factors. For example, epidermal growth factor (EGF) enhances osteogenic differentiation, while fibroblast growth factors and vascular endothelial growth factors stimulate blood vessel differentiation (angiogenesis).

In another example, the protein kinase C-binding protein NELL1 also known as NEL-like protein 1 (NELL1) or Nel-related protein 1 (NRP1) is a protein that in humans is encoded by the NELL1 gene. This gene encodes a cytoplasmic protein that contains epidermal growth factor (EGF)-like repeats. The encoded heterotrimeric protein may be involved in cell growth regulation and differentiation. A similar protein in rodents is involved in craniosynostosis. The administration of the protein NELL-1 intravenously stimulates significant bone formation through the regenerative ability of stem cells. The Nell-1 protein could further be applied or administered using other methods for other applications, such as coating medical devices. Vascular Endothelial Growth Factor (VEGF) is another example of a growth factor, also known as vascular permeability factor (VPF), is a signal protein produced by cells that stimulates the formation of blood vessels. To be specific, VEGF is a sub-family of growth factors, the platelet-derived growth factor family of cystine-knot growth factors. They are important signaling proteins involved in both vasculogenesis (the de novo formation of the embryonic circulatory system) and angiogenesis (the growth of blood vessels from pre-existing vasculature). Bone morphogenetic proteins (BMPs) are a group of growth factors also known as cytokines and as metabologens. Originally discovered by their ability to induce the formation of bone and cartilage, BMPs are now considered to constitute a group of morphogenetic signals, orchestrating tissue architecture throughout the body. The important functioning of BMP signals in physiology is emphasized by the multitude of roles for dysregulated BMP signaling in pathological processes. Cancerous disease often involves miss-regulation of the BMP signaling system. Absence of BMP signaling is, for instance, an important factor in the progression of colon cancer, and conversely, overactivation of BMP signaling following reflux-induced esophagitis provokes Barrett's esophagus and is thus instrumental in the development of adenocarcinoma in the proximal portion of the gastrointestinal tract.

A combination of a graphene coating and a growth factor coating helps reduce allergic reactions and rejection of a stent. Furthermore, the graphene coating helps functionalize the growth factor as it adheres much better to graphene than many of the metal alloys or polymers typically used in a stent. The graphene coating along had beneficial properties as it would prevent allergic reaction and rejection and provide a means for the growth factor coating to adhere. This combination also makes for a stent that can be used for a wider variety of applications such as Deep Vein Thrombosis (DVT) or revascularization rather than the more typical applications for Angioplasty and cardiac. Another embodiment and benefit is that the graphene layer could coat the entire stent or just portions of the stent depending on the need and application.

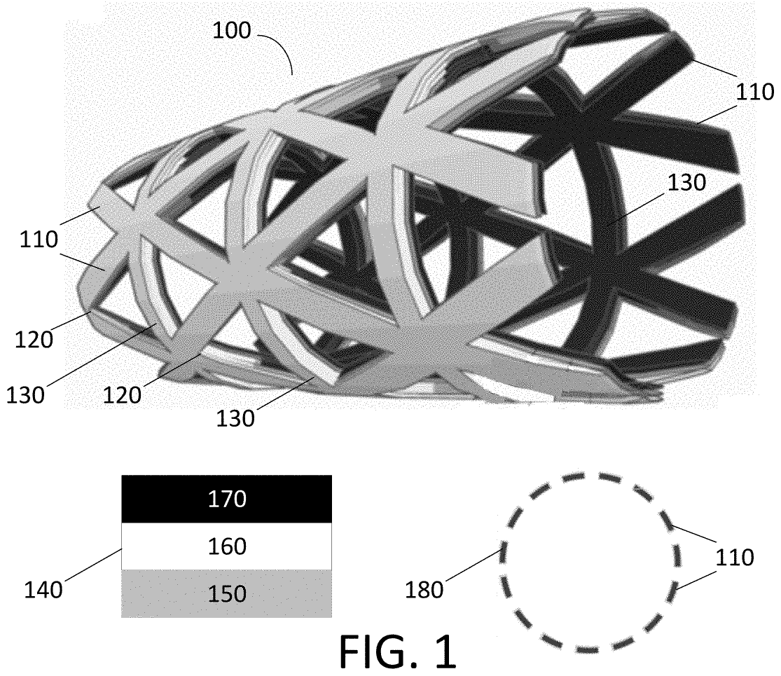

FIG. 1 is an apparatus for a stent with layer or layers of graphene and growth factors. This apparatus comprises of a stent. Stents are provided with scaffold structures which have low exposures (i.e. relatively few surfaces that touch tissue) when implanted in arteries and other blood vessels and lumens. The cross-sectional dimensions, materials, and patterns are controlled to provide sufficient strength and coverage while maintaining the low exposure. A stent that has low luminally exposed surface area and presents less foreign body material within a vessel. Furthermore, the stent's scaffold structural members, include struts 110 that may be configured to facilitate reduction of the exposed surface area of the stent. The structure of the stents are usually formed from a scaffold comprising a series of connected rings 120, each attached to struts 110, and crowns 130.

The rings 130 are typically connected by connectors (links) but in some cases crowns 120 may be connected directly to adjacent crowns to form the body of the stent. The scaffolds will usually be balloon expandable or the opposite and compressed to later be release and allowed to expand when in place, more usually being formed from a malleable metal. A metal scaffold, however, may be coated, covered, laminated with or otherwise joined to polymeric and other non-metallic materials. The scaffold may have an open cell structure, a closed cell structure, or a combination of both. Open and closed cell structures are well known and described in the literature of the art. A graphene coating which can cover all or only portions of the stent. The graphene coating possible embodied as glass, polymers (e.g., polystyrene, polydimethylsiloxane (“PDMS”), polycarbonate, polymethylmethacrylate, polyethylene, polypropylene, polyethylene terephthalate, polyvinyl chloride, styrene-ethylene/butylene-styrene, styrene-ethylene/propylene-styrene, polyurethane, silicone, etc.), metals and metal alloys (e.g., titanium, gold, platinum, silver, stainless steel, nitinol, cobalt-chrome, titanium alloys, stainless steel, tantalum, niobium, etc.), metal and metalloid oxides (e.g., aluminum oxide, zirconium oxide, silica, quartz, borosilicate, etc.), paper, plastics, various forms of carbon (e.g., diamond, graphite, black carbon, fullerene, nanotubes, graphene, diamond-like carbon, amorphous carbon, etc.) and other ceramic materials, composite materials and combinations of the above, and the like.

Graphene is a beneficial material for the use in medical, biomedical, drug delivery and in medical devices because of its unique physicochemical properties. In anticancer therapy, it also has an antimicrobial agent for bone and teeth implantation. The biocompatibility of the newly synthesized nanomaterials allows its substantial use in medicine and biology. The current review summarizes the chemical structure and biological application of graphene in various fields.

A growth factor coating such as, but not limited to NELL1 protein or vascular endothelial growth factor (VEGF). A growth factor such as NELL1 or VEGF would further help the regeneration of damaged tissue around the stent, help regenerate any tissue that was damaged during process of positioning the stent, further prevent the stent from being rejected or causing an allergic reaction. NELL1 or VEGF combined with the graphene layer or layers would greatly reduce many of the side effects related to current stent construction and placement. Furthermore, NELL1 and VEGF has a wide range of possible applications and could be used on several different types of stents for different medical conditionals such as Deep vein thrombosis (DVT) or angioplasty, at element 106. A close-up cross-sectional view of the stent with a layer or layers of graphene further coated or embedded with a growth factor such as, but not limited to the NELL1 protein or VEGF, at element 108. A method for developing a stent with a graphene coating as well as a grow factor, such as NELL1 protein or VEGF applied to the graphene. Where the graphene coating creates a biocompatible layer between the stent structure and tissue as well as an ideal material to embed or apply a growth factor at element 110. The graphene coating may be applied or modified by other deposition techniques, including but not limited to thermal atomic layer deposition (ALD), plasma ALD, photo-assisted ALD, metal ALD and catalytic ALD.

Item 140 of FIG. 1 illustrates a cross-section of scaffold or end view of a strut 110 of stint 100. This cross-sectional or strut end view illustrates several different layers 150, 160, and 170. Each of these layers may be comprised of different materials. For example, one of these layers may be a metal or polymer, a second layer may be graphene, and a third layer may be a growth factor. Item 180 of FIG. 1 is an end view if sting 100 that shows end portions of struts 110 when stint 100 has been expanded into a circular or cone shape.

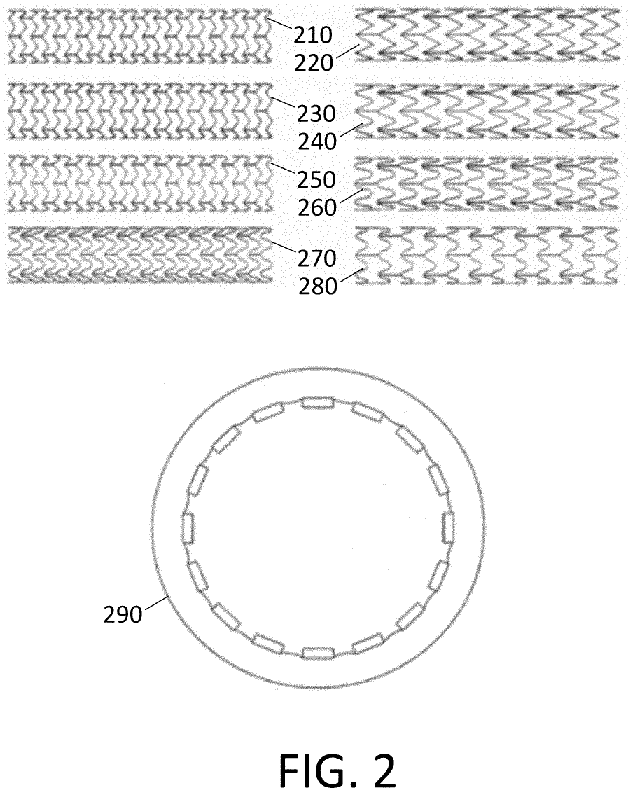

FIG. 2 illustrates structures of scaffold structures that may be used in different stints. The figure represents just a few exemplary stent scaffold structures 210, 220, 230, 240, 250, 20, 270, & 280. As described previously stents are provided with scaffold structures which as little exposures when implanted in arteries and other blood vessels and lumens. The cross-sectional dimensions, materials, and patterns are controlled to provide sufficient strength and coverage while maintaining the low exposure. The stent has low luminally exposed surface area and presents less foreign body material within a vessel. The structure of the stents are usually formed from a scaffold comprising a series of connected rings, each including struts and crowns as discussed in respect to FIG. 1. The rings are typically connected by connectors (links) but in some cases crowns may be connected directly to adjacent crowns to form the body of the stent. The scaffolds will usually be balloon expandable or the opposite and compressed to later be release and allowed to expand when in place, more usually being formed from a malleable metal. The metal scaffold, however, may be coated, covered, laminated with or otherwise joined to polymeric and other non-metallic materials. The scaffold may have an open cell structure, a closed cell structure, or a combination of both. Open and closed cell structures are well known and described in the patent and medical literature. An example of the placement of a stent is represented as item 290 of FIG. 2. One skilled in the art will recognize the stent had been expanded and portions of the stent are in contact with portions of the artery or vessel which was placed. This is significant, as the contact point can create a foreign body reaction, rejection or allergic reaction due to the materials stents are typically made.

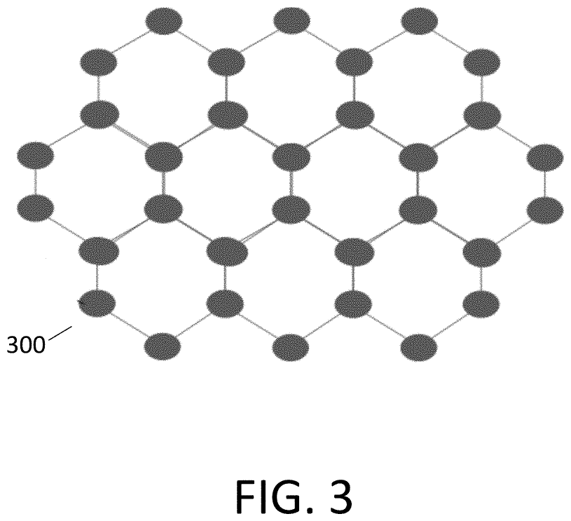

FIG. 3 illustrates a structure of a graphene layer that may be deposited on surfaces of a stint. This coating graphene can cover all or only some surfaces of the stent. As mentioned previously, the graphene coating 300 may vary depending on application and growth factor being applied. In some applications it only portions of the stent may need to be coated with graphene such as only the surfaces of the stent that would come in contact with tissue. In certain instances, all surfaces of the stent may be coated to help prevent adverse effects from the metallic or polymeric materials typically used in stents. Due to the biocompatibility and physiochemical properties of graphene, the use in medical, biomedical, drug delivery and in medical devices is compelling. The biomedical applications of graphene and its composite include its use in gene and small molecular drug delivery, specifically with regards to the use of a growth factor such as but not limited to NELL1 or VEGF. The biocompatibility and physiochemical properties of graphene alone would prove beneficial to stents but would also provide an ideal carrier for a drug coating such as a growth factor like NELL1 or VEGF. The combination of the two on a stent would further improve the stents adverse effects if placed alone.

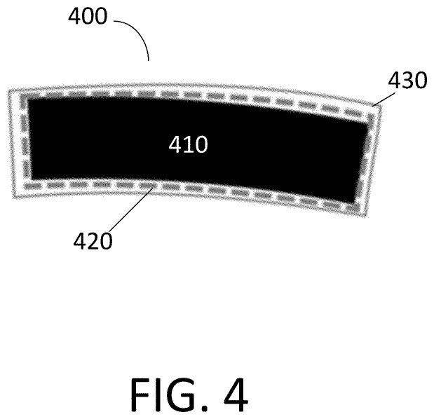

FIG. 4 illustrates a second cross-sectional or end view of a stint that is similar to the cross-sectional or end view 140 of stint 100 of FIG. 1. The cross-sectional view of stent portion 400 of FIG. 4 includes a base stent material 410, a graphene layer 420, and a growth factor coating 430. As described in FIG. 1, stents are provided with scaffold structures which have low exposures when implanted in arteries and other blood vessels and lumens. Stents are typically constructed of metallic or polymeric materials of a combination of both. The materials often have adverse side effects when they come in contact with human tissue, such as foreign body reaction, rejection or allergic reactions. The cross-sectional view of stent structure 400 is part of an expandable mesh is made of medical-grade base material that may be a metal such as, but not limited to, stainless steel or cobalt alloy metal, may be a polymeric material, or may be a nitinol stent frame with tantalum radiopaque markers at each end. Here again the layer or several layers of graphene 420 can cover all or only portions of the stent. As mentioned previously, the graphene layer or layers may consist of a mono-, bi- or multi-layers of graphene, specifically between 1 or 4 layers of graphene depending on the application and growth factor being applied.

In some applications, only portions of the stent may be coated with graphene, such as only the surfaces of the stent that would come in contact with tissue. In other instances, may be desirable to coat all surfaces of the stent to help prevent adverse effects from the metallic or polymeric materials typically used in stents. As describe in respect to FIG. 1, graphene is a beneficial material for the use in medical, biomedical, drug delivery and in medical devices because of its unique physiochemical properties. Nanomaterials such as graphene are important as they provide for the selective control of stem cell growth and differentiation, different nanoscale materials for stem cell control and differentiation. Furthermore, graphene and graphene oxide (GO) are used as scaffold materials for the culture of various stem cells. The growth, proliferation, and differentiation of different types of stem cells into specific tissue lineages have been shown to be supported and enhanced by the graphene-based nanomaterials.

A growth factor coating 430 may include, yet is not limited to the NELL1 protein. A growth factor such as NELL1 would further help the regeneration of damaged tissue around the stent, help regenerate any tissue that was damaged during process of positioning the stent, and may further prevent the stent from being rejected or causing an allergic reaction. NELL1 or VEGF combined with the graphene layer or layers would greatly many of the side effects related to current stent construction and placement. Furthermore, NELL1 has a wide range of possible applications and could be used on several different types of stents for different medical conditionals such as Deep vein thrombosis (DVT) or angioplasty. Other possible growth factors such as Vascular endothelial growth factor (VEGF) or other bone morphogenetic proteins (BMP) for the use of bone regeneration. The above mention growth factors or proteins could be used individually or in some instances in combination with one another.

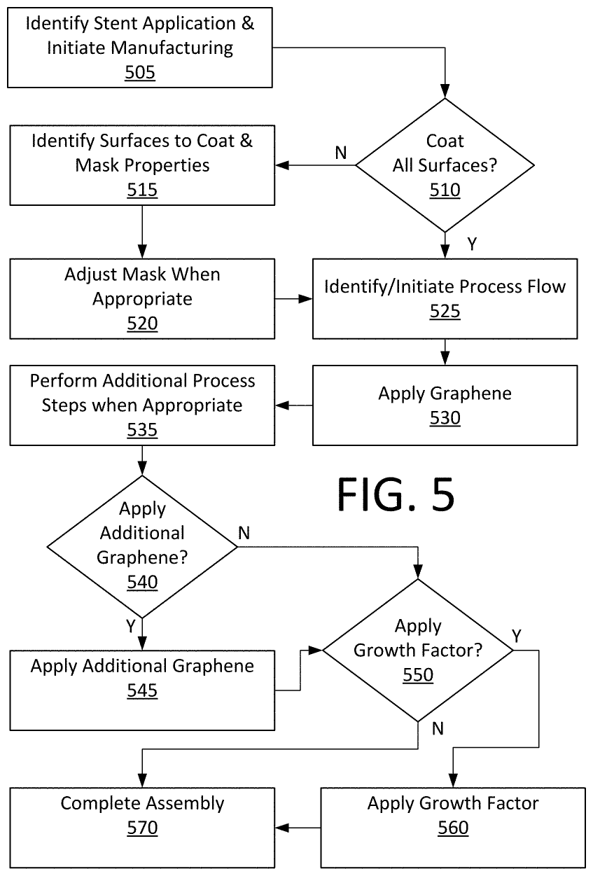

FIG. 5 illustrates a series of steps that may be performed when a stint consistent with the present disclosure is manufactured. FIG. 5 begins with step 505 where an application for the stint may be identified. This step may include collecting a set of specifications that may be specific to a location where the stent will be implanted in a person. In some instances, step 505 may include setting up a manufacturing line to manufacture types of stints meant to be implanted at specific locations of a person's body when stints are mass produced. Step 505 may be performed because requirements of manufacturing stints varies depending on the specific application of the stint or location where a stint will be placed.

Manufacturing processes of stints may vary based on a set of specifications or requirements that relate to locations in a body where a particular stint will be used. For example, a base portion of a stent could be created in step 505 using 3D printing methods for one application. Alternatively, a stent may be manufactured using a winding method where a wire mesh or structure is created by winding a wire around a jig, at step 505. The specification mentioned above may identify rules that identify locations on a stint where graphene should be deposited and these rules may identify a number of graphene layers (or thickness of graphene) to deposit on respective surfaces. In certain instances, some surfaces where graphene is deposited may include more layers (greater thickness) of graphene than other surfaces coated with graphene. These rules may also specify locations where certain types of growth factors should be deposited onto a stint.

Graphene coating processes may vary depending on how or where a particular stent will be applied. The aforementioned rules may specify which coating process should be used or a number of layers of graphene to use based on a location where a stint will be inserted. In some cases, stents require graphene to coat the entire surface of the stent structure, while other applications only require surfaces that will come in contact with tissue be coated with graphene. Because of this, determination step 510 may be performed to identify whether all surfaces of a stint will be coated with graphene. Depending on the application and the construction of the stent, graphene may be applied to all surfaces of the stent or applied to only specific surfaces. For example, only the surfaces that would come in contact with tissue may be coated with graphene and/or with a growth factor such as NELL1 or VEGF. When determination step 510 identifies that all surfaces of the stint should be not coated, program flow may move to step 515 of FIG. 1 where surfaces of the stint that should be coated with graphene may be identified. Step 515 may also include initiating a process of masking portions of the stint to prevent those portions from being coated by a coating process. This masking process may include adjusting the size of the mask in step 520 of FIG. 5. Such adjustments may include expanding or reducing (tightening) the size of the mask: this may be performed to compensate for different coefficients of expansion of a stint base material as compared to the masking material or to compensate for other issues that may affect edges of the mask.

When the interior surfaces of the stent are to be masked to prevent graphene from being applied the interior surfaces, a masking element maybe inserted in to the stent and expanded so that the masking elements comes into contact with the interior surface of the stent until it masks off the interior surfaces. This will prevent any material being applied to the stent from being applied to the interior surfaces. Furthermore, if the exterior surfaces of the stent need to be masked, the stent can be wrapped or inserted into a masking element which would be tightened until the masking element comes in contact with the exterior surfaces of the stent.

Once the mask and stent are in place the masking element may then expanded, such as inflating the element, or tightened or contracted around the stent, for example pulling the material tighter around the stent, using a vacuum, or applying heat to shrink the mask to come in contact with the stent surfaces.

When determination step 510 identifies that all surfaces of the stint should be coated or after step 520, program flow may move to step 525 where process steps relating to coating the stint or portions of the stint with graphene may include. Depending on a particular manufacturing process and coating specifications and corresponding rules for a particular stint, more than one graphene coating operation may be performed. In such an instance, the different graphene processing steps may include different methods for applying or depositing the graphene on the stint.

One example where the manufacturing of a stint could include multiple different graphene application processes may be when a copper substrate is coated with graphene before the graphene is transferred to a sheet of polymer via a hot rolling or pressure process. Such a process may include aligning a surface of the polymer and the side of a metallic substrate upon which graphene has been applied and then pressing these two surfaces together. This may include applying heat that may help facilitate transfer of the graphene onto the polymer. After the graphene has been transferred to the polymer, the resulting graphene coated polymer sheet may be cut into pieces or strips. Since edges of the cut surface would not include a graphene coating, these other surfaces may have to be coated with graphene using a secondary process that may be different than the first graphene coating process. This secondary process could include vapor deposition, for example.

After step 530 graphene may be applied to specific surfaces of a stint being manufactured. Here again this may be performed by various different methods. Next in step 535 additional process steps may be performed. Step 535 may include wrapping the polymer coated cut strips around a jig or form. Next determination step 540 may identify whether additional graphene should be applied to the stint, when yes program flow may move to step 545 where the additional graphene is applied to the stint.

The graphene coating process could be incorporated into the stent manufacturing process or could be a separate process. For example, maybe applied in the same manufacturing process where the graphene is applied during or after the stent is created. Alternatively, the graphene application process maybe separate from the stent manufacturing process, as stents may be acquired from a third party and then coated with a graphene.

To be able to select an application method of the graphene coating, the type of stent, its material make-up and the application may need to be known. The material make-up is especially important as different applications methods of graphene are required for different materials. All of this information is also required to determine how many layers are needed and will affect the method of adhering the graphene to the stent. For example, a chemical vapor deposition may be the ideal method of adhering graphene to type of material, while a lamination process or 3D printing process maybe better utilized for other materials. In another embodiment a combination of methods could also be used. Once the application and material of the stent is determined and the masks are in place and the method of applying the graphene coating is selected, the graphene layer or layers are then applied.

There are several methods for coating and adhering graphene to other materials and are well known in the art. For example, one possible method to coat a material with graphene is to use chemical vapor deposition (CVD). Furthermore, stents often are constructed of medical grade stainless steel. For example, a chemical vapor deposition method could be used to coat steel with graphene, where a single layer of graphene is grown on a stainless steel mesh, such as a stent, where the stainless steel is coated with Cu catalyst by using the chemical vapor deposition method. Even though the stainless steel consisted of different types of metals, such as Fe, Ni, and Cr, which can also be used as catalysts, the reason for coating Cu catalyst on the stainless steel surface had been related to the nature of the Cu, which promotes the growth of graphene with high quality and quantity at low temperature and time. In order to synthesize the graphene, a hydrocarbon source is required such as pure acetylene (C2H2), while carrier gas such as nitrogen (N2) and hydrogen (H2) can be utilized. Next, the reaction temperature and the time in the CVD technique can altered to grow the graphene depending on the required layer. It is then determined if the growth factor, such as NELL1 protein or VEGF, is applied to the same surfaces as the graphene or if it needs to be applied to all surfaces of the stent. If the growth factor only needs to be applied to the areas the graphene was applied, then the masks that were used for the graphene can remain in place. In other applications it may not matter if where the growth factor is coated in which the mask is removed and the growth factor is applied to the entire stent.

When determination step 540 identifies that additional graphene should be added to the stint or after step 545, program flow may move to determination step 550. Determination step 550 may identify whether a growth factor should be applied to the stint, when yes program flow may flow to step 560 where the growth factor is applied to the stint. Either when determination step 550 identifies that growth factor should not be applied to the sting or after step 560 of FIG. 5, program flow may move to step 570 where manufacturing or assembly of the stint may be completed. This may include one or more of cleaning the stint, placing stint in sterile packaging, and other steps.

Once a mask is no longer required it can be removed from the stent. The mask can be removed at any time after the graphene is applied. In some cases the mask may remain on until after the growth factor is applied. For example, if the mask is applied to the interior surfaces of the stent and was expanded, the mask would then be deflated or contracted. If the mask was on the exterior the mask it can be removed though any number of methods such as cutting or peeling it off.

Once the graphene has been applied, a growth factor, such as NELL1 protein or VEGF is then applied to the graphene surface. The functionalization of growth factors, such as NELL1 protein or VEGF, on to graphene is much more direct than putting it on other polymers. Furthermore, graphene's structure and biocompatibility make it an ideal material for applying growth factors. Specifically, because of its high surface area coupled with superior chemical stability, biocompatibility, and flexibility in functionalization render graphene-based nanomaterials one of the most exciting platforms for tissue engineering and regenerative medicine applications, especially for stem cell growth, proliferation, and differentiation. For example, the growth factor may be applied by coating of immersing the stent and graphene so that the growth factor is absorbed, or in another embodiment the growth factor is grown or cultured on the graphene on the stent. In another embodiment the growth factor is 3D printed on to the graphene on the stent. This would also allow for the growth factor to be applied specific regions of the stent rather than the entire stent.

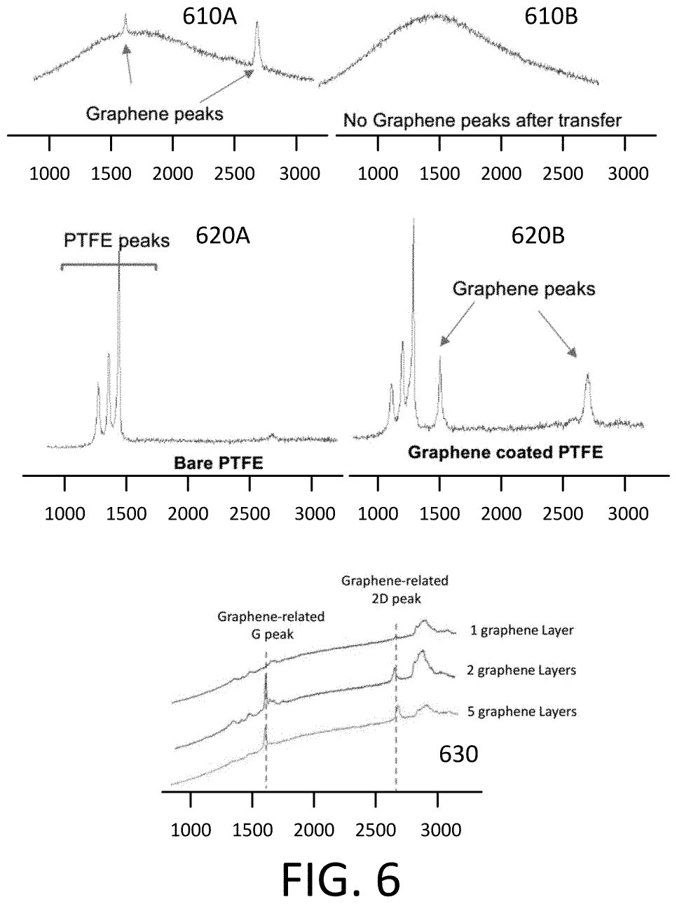

FIG. 6 illustrates several different measurements made by Raman spectroscopy. FIG. 6 includes curves 610A, 610B, 620A, 620B, and 630 that shows spectroscopic data show the successful transfer of graphene from a substrate to a sheet of PTFE. The horizontal axis included in FIG. 6 for each of curves 610A, 610B, 620A, 620B, and 630 show a measure of Raman shift in reciprocal centimeter (cm-1). Note that the horizontal scales rang from less than 1000 cm-1 to above 3000 cm-1.

Curve 610A shows spectroscopic data of a copper substrate that has graphene deposited on top of the copper substrate as indicated by the graphene peaks of curve 610A. Curve 610B shows spectroscopic data of the same copper substrate after the graphene has been moved to the sheet of PTFE. Note that curve 610B does not include the graphene peaks of curve 610A, this is because the graphene has been moved to a sheet of PTFE.

Curve 620A shows spectroscopic data of a bare sheet of PTFE before graphene has been transferred to the PTFE sheet. Curve 620B shows spectroscopic data of the sheet of PTFE after the graphene has been transferred to the PTFE sheet. Both curve 620A and curve 620B include a series of PTFE peaks located in the range of about 1200-1500 cm-1. These curves show that the graphene has been transferred from the copper substrate to the PTFE. The graphene was moved from the copper substrate to the sheet of PTFE using a process similar to the hot press process discussed above.

The three curves 630 at the bottom of FIG. 6 illustrates spectroscopic data of three different transfers of graphene to a PTFE sheet from a copper substrate. Here, the top curve shows a transfer of one layer of graphene, the middle curve shows a transfer of two graphene layers, and the bottom curve shows a transfer of five graphene layers. While it is difficult to see the graphene peaks in the top curve of curves 630, higher resolution data shows that the graphene did transfer. Note that the height of respective graphene peaks increase with the number of graphene layers. Experiments have shown that increasing an number of layers of graphene on a copper substrate reduces resistance of the surface of the graphene on copper surface. Measurements have shown resistances of one to two thousand (1-2 K) ohms of resistance per unit area when one graphene layer was used, two graphene layers resulted in resistances of 0.5 K to 0.8 K ohms, and five layers of graphene resulted in resistances of 0.2 to 0.3 K ohms.

What is claimed is:

1. A stint for insertion into a person, the stint comprising:
a base material included in a structure of the stint; and
a biocompatible graphene that is deposited onto a surface of the structure according to a rule, wherein the structure of the stint is capable of expanding to a larger size from a compressed size after being inserted into a body of a patient and the stint is received by the body of the patient based on the biocompatible graphene being deposited according to the rule.

2. The stint of claim 1, further comprising a growth factor deposited onto the surface or a second surface of the stint.

3. The stint of claim 1, wherein the base material includes a polymer.

4. The stint of claim 1, wherein the polymer is polyurethane (PU).

5. The stint of claim 1, wherein the polymer is PU disposed on polytetrafluoroethylene (PTFE).

6. The stint of claim 1, wherein the PTFE is expanded PTFE (ePTFE).

7. The stint of claim 1, wherein the base material is formed into the structure and the structure has a scaffold shape when expanded.

8. The stint of claim 1, wherein the base material is a metal.

9. A method for making a stint, the method comprising:
preparing a base material to deposit graphene onto;
identifying a method for depositing the graphene onto a portion of the base material; and
depositing the graphene onto the portion of the base material via the identified method.

10. The method of claim 9, further comprising identifying a location in the body of the patient where the stint will be inserted, wherein the portion of the base material that the graphene is deposited is based on a rule associated with the location in the body of the patient.

11. The method of claim 9, further comprising depositing a growth factor onto the portion of the stint or a second portion of the stint.

12. The method of claim 9, further comprising:
aligning a substrate that includes the graphene in proximity to the base material;
pressing the substrate onto the base material;
heating the substrate to a temperature; and
removing the substrate, wherein the removal of the substrate results in the graphene being deposited on the base material based on the heating of the substrate and the pressing of the substrate onto the base material.

13. The method of claim 12, further comprising depositing the graphene onto the substrate.

14. The method of claim 13, wherein the substrate includes copper.

15. The method of claim 12, further comprising changing the temperature of the substrate to a second temperature, wherein the deposition of the graphene on the base material is also based on the changing of the temperature of the substrate to the second temperature.

16. The method of claim 9, further comprising:
adding a masking material to a part of the base material; and
removing the masking material from the part of the base material after the graphene is deposited onto the portion of the base material.

17. The method of claim 13, further comprising depositing a growth factor onto a selected part of the base material or the deposited graphene.

18. The method of claim 9, further comprising depositing additional graphene onto one or more parts of the base material or the previously deposited graphene.

19. The method of claim 9, further comprising forming the base material into a shape of the stint.

20. The method of claim 9, further comprising receiving the stint from a manufacturer before depositing the graphene onto the portion of the base material of the stint.

21. The method of claim 9, further comprising preparing to deposit the graphene via a vapor deposition process, wherein the graphene is deposited onto the portion of the base material via the vapor deposition process.
